(19) United States
(12) Patent Application Publication
(10) Pub. No.: US 2023/0330377 A1
(43) Pub. Date: Oct. 19, 2023

(54) METHOD AND APPARATUS FOR MONITORING A NASAL HIGH-FLOW THERAPY SYSTEM

(71) Applicant: UNIVERSITEIT TWENTE, ENSCHEDE (NL)

(72) Inventors: Rob HAGMEIJER, ENSCHEDE (NL); Rutger Hendrik Johan HEBBINK, ENSCHEDE (NL)

(73) Assignee: UNIVERSITEIT TWENTE, ENSCHEDE (NL)

(21) Appl. No.: 18/028,947

(22) PCT Filed: Sep. 27, 2021
(86) PCT No.: PCT/NL2021/050583

§ 371 Date: Mar. 28, 2023

(30) Foreign Application Priority Data
Sep. 28, 2020 (NL) 2026554

(51) Int. Cl. A61M 16/06

(52) U.S. Cl. CPC A61M 16/0672; A61M 2016/0027

(57) ABSTRACT

A method for monitoring a system for injecting heated air into a patient. The system includes a source of air, a device for heating the air, a cannula for insertion into nostrils of the patient, a first conduit interconnecting the source and the device, and a second conduit interconnecting the device and the cannula. The method includes deriving pressure in the cannula from pressure of the air between the source and the cannula. The method includes measuring a pressure of the air at between the source and the cannula, determining a flow rate of the air in the system, deriving a first function representative of a respiratory flow rate of the patient from the measured pressure and the system air flow rate, deriving a second function representative of a respiratory volume of the patient from the measured pressure and the system air flow rate, and graphically displaying the functions.

The invention relates to a method for monitoring a system for injecting heated air into a patient, wherein the system includes a source of air, a device for heating the air, a cannula for insertion into nostrils of the patient, a first conduit interconnecting the source and the device, and a second conduit interconnecting the device and the cannula. Injecting heated air into a patient’s nostrils is known as Nasal High-Flow (NHF) therapy. As used in this application, the term “air” may denote either ambient air having an oxygen content of approximately 21 percent, or pure oxygen, or any mixture of ambient air and oxygen. This “air” may be provided by an external source, typically an air/oxygen system of a hospital. Besides being heated, the air may also be humidified if so required. However, this depends on the specific application and is not essential.

NHF therapy consists of injecting the heated and oxygenized (and possibly humidified) air at relatively high flow rates into the nostrils of a patient through a loosely fitted nasal cannula. It is applied to neonates, infants and adults with acute hypoxemic respiratory failure, but the conjectured working principles, i.e. an increase in average airway pressure and an increase in wash-out, have not been proven yet. The protocol followed by physicians mainly consists of applying a fixed flow rate which is determined by the size of the cannula used and by the body weight of the patient. During therapy the patient is monitored by means of separate sensors that measure heart rate, respiratory rate, and oxygen saturation. Pressures and respiratory patterns are not measured at all, and the flow rate is manually set by means of visual reading.

The invention has for its object to provide an improved method for monitoring a system for NHF therapy.

In accordance with a first aspect of the invention, this is achieved by a method in which a pressure in the cannula is derived from a pressure of the air measured at the first location between the source and the cannula. The pressure in the cannula is the pressure at which the air enters the patient’s nostrils. This pressure should be higher than the ambient pressure if it is to be ensured that inhaled air comes from the device exclusively, and not from ambient air.

In an embodiment of the method, a flow rate of the air in the system may be determined and the pressure in the cannula may be derived from the measured pressure at the first location by compensating for the determined flow rate. Thus, the pressure in the cannula can be determined on the basis of functions which can easily be measured.

Compensating for the flow rate may include scaling the flow rate by a function of the Reynolds number of the flow of air through the system. The Reynolds number may be derived from a pressure differential between the measured pressure at the first location and a measured pressure at the second location upstream of the first location while a flow restriction is present between the first and second locations. The presence of the flow restriction leads to a measurable pressure differential which may form the basis for all further derivations. The scaling step results in a non-dimensional ratio of flow resistances.

In an embodiment of the invention, the derived pressure in the cannula may be displayed, so that an operator can check the functioning of the system at a glance.

In a further embodiment, the derived pressure in the cannula may be compared to a target pressure to define an error signal, and a warning signal may be generated when the error signal exceeds a predetermined threshold. The warning signal may be an audible signal like a buzz or beep, a visible signal like a flashing light, or a combination of such signals. In this way an operator may be warned that the system is deviating from a user-defined target pressure, and may make adjustments. A flow rate of the air from the high-flow device into the nostrils of the patient P should be greater than the flow rate inhaled by the patient, so as to ensure that the patient only inhales the air provided by the high-flow device, rather than ambient air. On the other hand, the air pressure in the cannula should not be so high that it might cause damage to parts of the patient’s respiratory tract. Therefore, the pressure in the cannula must be maintained within a range of safe and effective values between a lower limit and an upper limit.

In a specific embodiment of the method, the derived pressure in the cannula may be compared to ambient pressure and a warning signal may be generated when the derived pressure in the cannula is below ambient pressure. For the NHF therapy to be effective, it is especially important that the pressure of the air in the cannula is greater than ambient pressure pₐₜₘ, so as to ensure that the air flowing through the cannula forms a jet that enters the nasal cavity of the patient. The formation of such a jet is an important factor in inducing the so-called “wash-out effect”, i.e. the rapid replacement of air exhaled by the patient with air provided by the high-flow device. If the cannula air pressure drops below ambient pressure, this is an indication that the wash-out effect will not occur, which in turn means that the NHF therapy may be ineffective and the patient may be inhaling ambient air.

In an embodiment of the method, the error signal may be used for automatically adjusting the flow of air through the system. The error signal may for instance be communicated as feedback to a valve arranged somewhere between the source of air and the cannula, thus resulting in a system which is pressure controlled, rather than flow-rate controlled as conventional systems are.

In accordance with a further aspect of the invention, a method is provided which comprises the steps of:
continuously or periodically measuring a pressure of the air at a first location between the source and the cannula,
determining a flow rate of the air in the system,
deriving a first function representative of a respiratory flow rate of the patient from the measured pressure and the system air flow rate,
deriving a second function representative of a respiratory volume of the patient from the measured pressure and the system air flow rate, and
displaying the first and second functions and/or a variable derived therefrom.

In this way the patient’s respiratory pattern may be measured on the basis of a non-invasive pressure measurement. The breathing pattern or a variable derived from the first and second functions may be displayed. Displaying the first and second functions may provide a graphical representation of the breathing pattern. This graphical representation may be used to determine if the settings of the system need to be adjusted and may eventually be used to determine when the therapy can be ended and the system can be switched off. An example of a variable that may be derived from the first and second functions to be displayed is FEV1, which is an important parameter for characterizing a patient’s breathing.

The pressure may be measured during a plurality of respiratory cycles of the patient. This allows the breathing pattern to be determined more accurately.

In an embodiment which is computationally simple, the second function may be derived by integrating the first function. Since the respiration volume is related to a time-integral of the respiratory flow rate, this derivation yields a true representation of the breathing pattern.

In a further embodiment the first and second functions may be rendered non-dimensional. By using non-dimensional functions a generalized representation of the respiratory cycle may be presented, which allows swift and easy comparisons with representations relating to other patients or other circumstances.

The first and second functions may be rendered non-dimensional by the steps of measuring the pressure when no air flows in the system, measuring the pressure while air is flowing in the system, and dividing results of the pressure measurements while air is flowing by results of the pressure measurements when no air flows.

In order to allow the functioning of the system to be monitored efficiently, the first function and second function may be displayed in a single graph. Such a single graph gives a clear representation of the patient’s breathing pattern and may be interpreted at a glance.

The steps of deriving, displaying and comparing the pressure in the cannula and/or deriving and displaying the first and second functions may be performed by a computer.

The method described above allows pressures and flow rates to be determined independently of the geometry of the flow channel, which is important since this geometry is defined to a large extent by the shape of the patient’s nostril. The method involves the use of pressure data collected in a wide interval around a specific time instant for computing a ratio of aerodynamic inspirational resistance to aerodynamic expirational resistance. In this way the method is self-calibrating. Eventually, the method leads to the derivation of a case-specific ratio of non-linear aerodynamic resistances for inspiration and expiration. As such, the method is independent of an orientation and shape of the cannula, and independent of the patient.

The invention also relates to an apparatus with which the above method may be carried out. In accordance with an aspect of the invention, such an apparatus for monitoring a system for injecting heated air into a patient may comprise a cannula pressure derivation module in communication with a pressure measuring element arranged at a first location between the source and the cannula and configured for deriving a pressure in the cannula from the measured pressure at the first location.

In accordance with a further aspect of the invention, such a monitoring apparatus may comprise:
a pressure measuring element arranged at a first location between the source and the cannula and configured for continuously or periodically measuring a pressure of the air at the first location,
a flow rate determination element for determining a flow rate of the air in the system,
a first derivation module in communication with the pressure measuring element and the flow rate determination element, and configured for deriving a first function representative of a respiratory flow rate of the patient from the measured pressure and the system air flow rate,
a second derivation module in communication with at least one of the pressure measuring element, the flow rate determination element and the first derivation module, and configured for deriving a second function representative of a respiratory volume of the patient from the measured pressure and the system air flow rate, and
a display module in communication with the first and second derivation modules and configured for displaying the first and second functions and/or a variable derived therefrom.

Further embodiments of the monitoring apparatus of the invention are defined in the dependent claims 17-23 and 25-29.

The invention will now be elucidated in the following detailed description of an exemplary embodiment thereof, with reference being made to the annexed drawings, in which:

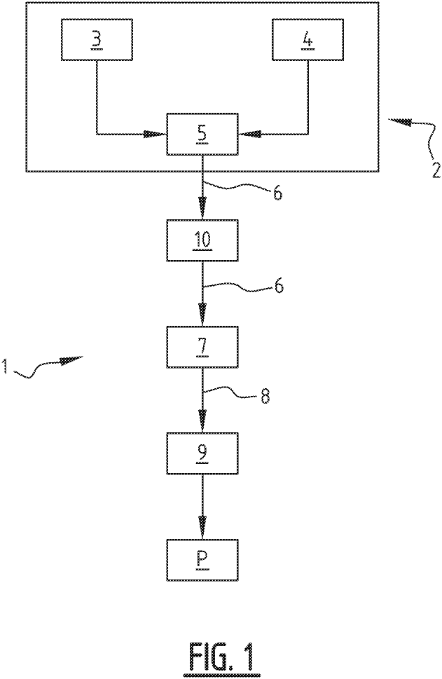

FIG. 1 is a schematic representation of a system for NHF therapy including a monitoring apparatus in accordance with the invention;

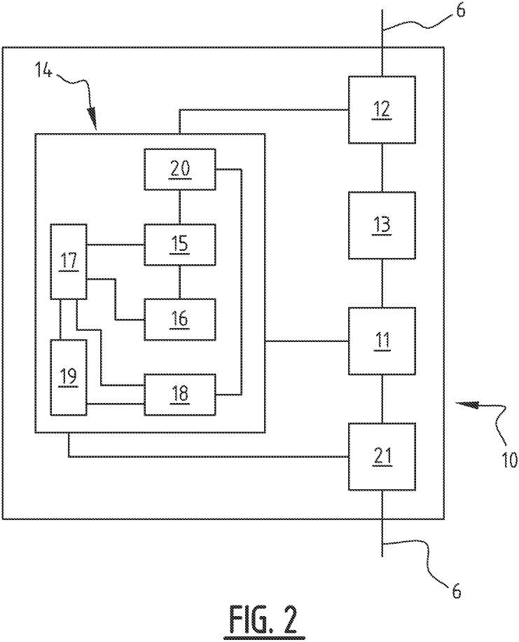

FIG. 2 is a schematic representation of the monitoring apparatus;

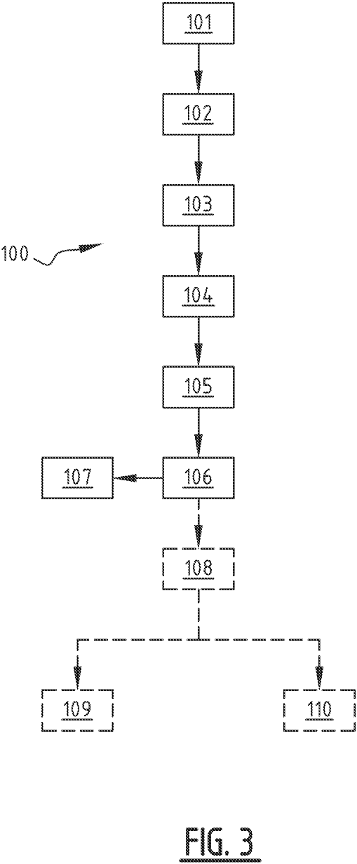

FIG. 3 shows the steps of a method in accordance with the invention which lead to derivation of the pressure in the cannula;

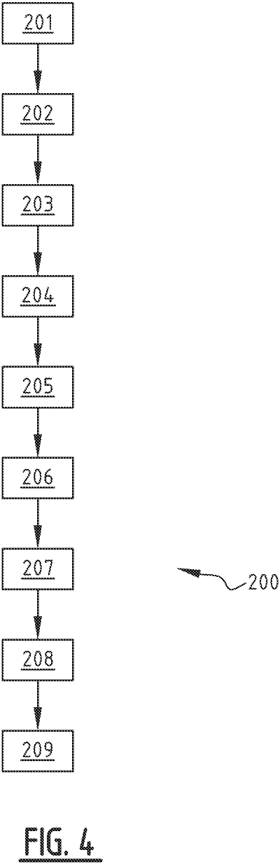

FIG. 4 shows the steps of the method of the invention which lead to derivation of a graphical representation of first and second functions representative of a breathing pattern of a patient;

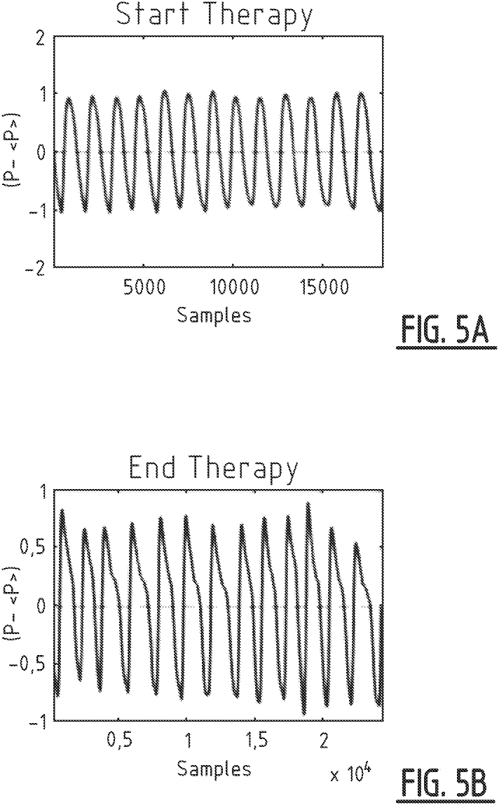

FIGS. 5A and 5B show pressure signals as a function of time at the start and at the end of an NHF therapy; and

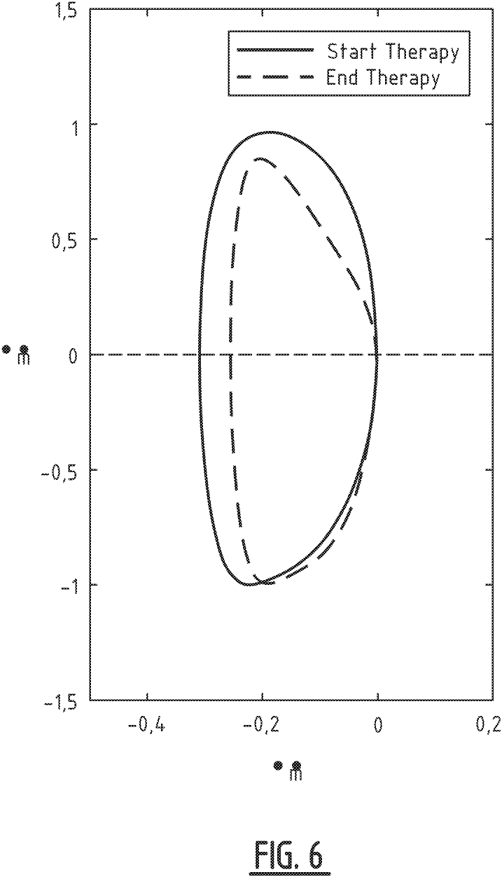

FIG. 6 shows a graphical representation of a breathing pattern of a patient at the start and at the end of the NHF therapy.

A system 1 for NHF therapy comprises a source of air 2, which in the illustrated example includes a source of ambient air 3, a source of oxygen 4 and an adjustable mixer 5 (FIG. 1). A first conduit 6 connects the source of air 2 with a device 7 for heating the air. In the illustrated example the heating device 7 may further be configured for humidifying the air as well, but this is not essential. From this device 7 a second conduit 8 leads to a cannula 9, which may be loosely inserted into nostrils of a patient P. Located between the source 2 and the device 7 is an apparatus 10 for monitoring the system 1.

The monitoring apparatus 10 comprises a first pressure measuring element or pressure sensor 11 and a second pressure measuring element or pressure sensor 12 arranged upstream of the first pressure sensor 11 (FIG. 2). A restriction 13 is arranged between the first and second pressure sensors 11, 12. Since in the illustrated embodiment the monitoring apparatus 10 is arranged upstream of the humidifying and heating device 7, the first and second pressure sensors 11, 12 measure pressures of the air while it is still dry and at ambient temperature.

It is also conceivable that the humidifying and heating device 7 is supplied with ambient air that is drawn in from the surrounding atmosphere, rather than being connected to the source 2. In that case the monitoring apparatus 10 may be arranged downstream of the humidifying and heating device 7 and the first and second pressure sensors 11, 12 may measure the pressure of the warm and humid air that is supplied to the patient.

In fact, the exact locations of the first and second pressure sensors between the source of air 2 and the cannula 9 are not important, as long as pressure differential can be measured between two locations in the air supply to the patient.

The monitoring apparatus 10 further comprises a processing unit 14 which is connected to both the first and second pressure sensors 11, 12. The processing unit 14 includes a flow rate determination element 20, a first derivation module 15, a second derivation module 16 and a display module 17. The first derivation module 15 is in communication with the first pressure sensor 11 and the flow rate determination element 20, and the second derivation module 16 is in communication with the first pressure sensor and with the first derivation module 15. The display module 17 is in communication with both the first and the second derivation modules 15, 16.

The first derivation module 15 is configured for deriving a first function representative of a respiratory flow rate of the patient P from a pressure p_{b}(t) measured by the first pressure sensor 11, i.e. the pressure downstream of the flow restriction 13, and from the flow rate Q of the air in the system 1. The second derivation module 16 is configured for deriving a second function representative of a respiratory volume of the patient P from the measured pressure p_{b}(t) and the flow rate Q. The display module 17 is configured for displaying the first and second functions in a graphical representation.

The monitoring apparatus 10 further comprises a cannula pressure derivation module 18. The cannula pressure derivation module 18 is in communication with the first and second pressure measuring elements or pressure sensors 11, 12 and with the flow rate determination element 20. This module 18 is configured for deriving a pressure p_{c} in the cannula 9 from the pressure p_{b} measured by the first pressure sensor 11. The derivation of the cannula pressure p_{c} also involves the pressure pₐ measured by the second pressure sensor 12, as well as the air flow rate Q in the system 1, as will be explained below. The cannula pressure derivation module 18 is in communication with the display module 17 so that the derived pressure p_{c} in the cannula 9 may be displayed.

The monitoring apparatus 10 also comprises a comparator 19 which is in communication with the cannula pressure derivation module 18. In the comparator 19 the derived pressure p_{c} in the cannula 9 is compared to a target pressure p_{tg} to define an error signal Δp. A warning signal is generated when the error signal has a magnitude which exceeds a predetermined threshold. This error signal Δp may be used in a feedback loop to control the flow through the system 1. To that end the monitoring apparatus 10 includes a flow control member or valve 21 which is in communication with the comparator 19 and which serves to limit or increase the flow of air through the first conduit 6 on the basis of the error signal. In particular the comparator 19 may be configured to compare the derived pressure p_{c} in the cannula 9 to ambient pressure pₐₜₘ. If the derived pressure p_{c} in the cannula 9 is found to be below ambient pressure pₐₜₘ, this is an indication that the cannula flow fails to form a jet entering the nasal cavity of the patient P. This in turn means that no wash-out effect is induced, so that the therapy will not be effective.

The monitoring apparatus 10 is used to monitor the flow rate Q of air through the system 1, the pressure at the cannula exit p_{c}, the respiratory rate of the patient P and the patient’s breathing pattern on the basis of continuous or periodic measurements of the air pressures pₐ and p_{b} upstream and downstream, respectively, of the flow restriction 13 by the first and second pressure sensors 11, 12. The relationships between these various functions are explained below.

### GENERAL PRESSURE-FLOW RATE RELATION

From the theory of fluid mechanics it follows that the difference between the two pressures p₁ and p₂ at the beginning and at the end of a flow channel of arbitrary shape, can be written in the form

$${p}_{1} - {p}_{2} = \frac{1}{2} F \left(Re\right) \frac{ρ {Q}^{2}}{{A}^{2}} \qquad \text{(1)}$$

where A is a characteristic cross-section of the channel, p is the mass density, Q is the flow rate through the channel, and Re is a non-dimensional ratio of these functions called the Reynolds number. The Reynolds number is defined as:

$$R e = \frac{2}{\sqrt{π}} \frac{ρ Q}{μ \sqrt{A}} \qquad \text{(2)}$$

where µ is the dynamic viscosity. The function F (Re) is non-dimensional and can be derived either theoretically or experimentally. For laminar Hagen-Poiseuille flow in a circular channel of diameter D and length L one finds for example that

$${F}_{H P} \left(R e\right) = \frac{64}{R e} \frac{L}{D} \qquad \text{(3)}$$

When, for a given channel with known A, and a given fluid with density ρ and viscosity µ, both the flow rate Q and the pressure difference (pₐ - p_{b}) are measured in an experiment, one can compute the Reynolds number from Eq. (2) and the corresponding function value F (Re) as:

$$F \left(R e\right) = \frac{2 {A}^{2} \left({p}_{1} - {p}_{2}\right)}{ρ {Q}^{2}} \qquad \text{(4)}$$

### DETERMINATION OF FLOW-RATE

To determine the time dependent flow rate through the NHF system 1 the above Eq. (1) must be solved for Q, with p₁ = pₐ, the pressure at the entrance of the monitoring apparatus 10 as measured by the second pressure sensor 12, and p₂ = p_{b}, the pressure at the exit of the monitoring apparatus 10, downstream of the flow restriction 13, as measured by the first pressure sensor 11:

$$Q = \sqrt{\frac{2 {A}^{2} \left({p}_{a} - {p}_{b}\right)}{ρ {F}_{m} \left(R e\right)} ,} R e ≡ \frac{2}{\sqrt{π}} \frac{ρ Q}{μ \sqrt{A}} \qquad \text{(5)}$$

where A is a characteristic cross-sectional area of the first conduit 6 in the monitoring apparatus 10. The corresponding function Fₘ (Re) is determined in advance in a laboratory experiment by using Eq. (4) for a range of flow rates.

### DETERMINATION OF CANNULA EXIT PRESSURE

To determine the pressure p_{c} of the air in the cannula 9, in particular the pressure at which the air exits the cannula, Eq. (1) can be applied with p₁, = p_{b}, the pressure measured by the first pressure sensor 11 at the exit of the monitoring apparatus, downstream of the flow restriction 13, and Q determined with the procedure described above:

$${p}_{c} = {p}_{b} - \frac{1}{2} {F}_{c} \left(R e\right) \frac{ρ {Q}^{2}}{{A}^{2}} \qquad \text{(6)}$$

The corresponding function F_{c} (Re) is determined prior to connecting the NHF system 1 to the patient P by using Eq. (4) for a range of flow rates. In other words, the monitoring apparatus 10 can calibrate itself for each new combination of NHF system 1 and cannula 9.

The above-described steps of the method 100 for deriving the cannula pressure p_{c} from the pressure p_{b} measured downstream of the flow restriction 13 are illustrated in FIG. 3.

In step 101 the combination of the NHF system 1 and the cannula 9 is calibrated by determining the non-dimensional function F_{c} (Re) for the combination of the monitoring apparatus 10 and the cannula 9 for a range of flow rates Q by using Eq. (4).

Then the pressure p_{b} of the air exiting the monitoring apparatus 10 through the first conduit 6, i.e. after passing through the flow restriction 13, but in this embodiment still upstream of the humidifying and heating device 7, is measured by the first pressure sensor 11 in step 102.

In step 103 the pressure pₐ of the air flowing at the entry of the monitoring apparatus 10, i.e. upstream of the flow restriction 13 is measured by the second pressure sensor 12.

In step 104 the non-dimensional function Fₘ (Re) for the monitoring apparatus 10 is determined experimentally, e.g. in a laboratory, for a range of flow rates Q by using Eq. (4).

Subsequently, in step 105 the actual flow rate Q is determined from Eq. (5) by substituting the pressures p_{b} and pₐ measured in steps 102 and 103 and the non-dimensional function Fₘ (Re) determined in step 104.

And finally, in step 106 the cannula exit pressure p_{c} is determined from Eq. (6) by substituting the pressure p_{b} measured in step 102, the non-dimensional function F_{c} (Re) determined in step 101 and the flow rate Q determined in step 105.

The cannula exit pressure p_{c} determined in step 106 may be displayed by the display module 17 (step 107).

Optionally, the cannula exit pressure p_{c} determined in step 106 can be compared to a target pressure p_{tg} entered into the system 1 by a user (step 108) to define an error signal Δp. This error signal may be compared to a threshold, and when it exceeds the threshold a warning signal may be generated (step 109). For the NHF therapy to be effective, it is especially important that the cannula exit pressure p_{c} is greater than ambient pressure pₐₜₘ, so that the flow of humidified and heated air reaches the patient’s respiratory tract. The flow rate Q from the NHF system 1 into the nostrils of the patient P should be greater than the flow of ambient air inhaled by the patient. On the other hand, the cannula exit pressure p_{c} should not be so high that it might cause damage to parts of the patient’s respiratory tract. Therefore, the cannula exit pressure p_{c} must be kept between a lower limit and an upper limit.

To this end, the error signal Δp may be used in a feedback loop to a flow control member, e.g. a controllable valve, in order to establish pressure control of the NHF therapy (step 110).

The monitoring apparatus 10 not only determines the cannula exit pressure p_{c}, but may also determine the respiratory flow rate of the patient and establish a breathing pattern. The steps of the method 200 that is used to this end are shown in FIG. 4:

### DETERMINATION OF RESPIRATORY FLOW RATE

To determine the respiratory flow rate at given time t, in step 201 the pressure p_{b}(t) as measured by the first pressure sensor 11 is monitored and translated into the cannula pressure p_{c}(t) by means of equation (6). In step 202 the pressure difference Δp(t) is defined as the difference between the cannula pressure p_{c}(t) and the ambient pressure pₐₜₘ:

$$Δ p \left(t\right) ≡ {p}_{c} \left(t\right) - {p}_{a t m} \qquad \text{(7)}$$

Firstly, in step 203 the device flow is switched off (Q_{d}= 0) and for a number of respiration cycles K, with K ≈ 10, pressures p(t) are recorded and integrated as follows (step 204):

$$\left〈{I}_{i n}^{0}\right〉 ≡ {{\int }_{0}^{T} min \left(0 , \text{sign} \left(Δ p\right)\right) \left|Δ p\right|}^{{b}_{i n}} \text{d} τ \leq \text{0} \qquad \text{(8)}$$

$$\left〈{I}_{e x}^{0}\right〉 ≡ {{\int }_{0}^{T} max \left(0 , \text{sign} \left(Δ p\right)\right) \left|Δ p\right|}^{{b}_{e x}} \text{d} τ \leq \text{0} \qquad \text{(9)}$$

In these expressions, bᵢₙ and bₑₓ are given constants for inhalation and exhalation, respectively, and T is defined as the time needed for the K respiration cycles to be completed. The constants, bᵢₙ and bₑₓ both depend on the sign of Δp, which reflects that the aerodynamic resistance not only depends on the geometry of the flow channel, but also on the direction of the flow and on the upstream flow condition. For example, when the flow is fully laminar b=1, and when the flow is fully turbulent b= ½.

Secondly, in step 205 the device flow is switched on (Q_{d} > 0), and for a number of respiration cycles K, with K ≈ 10, pressures p(t) are again recorded and integrated (step 206):

$$\left〈{I}_{i n}\right〉 ≡ \frac{1}{K} {{\int }_{0}^{T} min \left(0 , \text{sign} \left(Δ p\right)\right) \left|Δ p\right|}^{{b}_{i n}} \text{d} τ \leq \text{0} \qquad \text{(10)}$$

$$\left〈{I}_{e x}\right〉 ≡ \frac{1}{K} {{\int }_{0}^{T} max \left(0 , \text{sign} \left(Δ p\right)\right) \left|Δ p\right|}^{{b}_{e x}} \text{d} τ \leq \text{0} \qquad \text{(11)}$$

It should be noted that steps 203 and 204 could be performed after steps 205 and 206; the order is not important, as long as there are pressure measurements with the air flow in the system switched on and with the air flow switched off.

In step 207 the non-dimensional airway flow rate Q̅ₐ, taken positive when exhaling, is computed from the recorded pressure and the expressions (8) - (11) above as:

$${\tilde{Q}}_{a} \left(t\right) = \left\{\begin{matrix}\frac{{\left|Δ p\right|}^{{b}_{i n}} \left〈T\right〉}{\left〈{I}_{i n}^{0}\right〉} - \left(\frac{\left〈{I}_{e x}\right〉}{\left〈{I}_{e x}^{0}\right〉} - \frac{\left〈{I}_{i n}\right〉}{\left〈{I}_{i n}^{0}\right〉}\right) , Δ p < 0 \\ \frac{{\left|Δ p\right|}^{{b}_{e x}} \left〈T\right〉}{\left〈{I}_{e x}^{0}\right〉} - \left(\frac{\left〈{I}_{e x}\right〉}{\left〈{I}_{e x}^{0}\right〉} - \frac{\left〈{I}_{i n}\right〉}{\left〈{I}_{i n}^{0}\right〉}\right) , Δ p < 0\end{matrix}\right) \qquad \text{(12)}$$

where (T) ≡ T/K.

Alternatively, a non-breathing pressure p_{∗} can be determined by letting the patient temporarily breathe through the mouth, by analyzing the pressure signal such as for example by performing any sort of averaging operation, or by using an additional external measurement. Formula (12) for computing the non-dimensional airway flow rate Q̅ₐ may be then be modified as follows:

$${\tilde{Q}}_{a} \left(t\right) = \left\{\begin{matrix}\frac{{\left|p \left(t\right) - {p}_{∗}\right|}^{{b}_{i n}} \left〈T\right〉}{\left〈{I}_{i n}\right〉} , Δ p < 0 , \\ \frac{{\left|p \left(t\right) - {p}_{∗}\right|}^{{b}_{e x}} \left〈T\right〉}{\left〈{I}_{e x}\right〉} , Δ p < 0.\end{matrix}\right) \qquad \text{(12a)}$$

where 〈Iᵢₙ〉 and 〈Iₑₓ〉 also have been calculated with Δp = p - p_{∗}.

And finally, in step 208 the non-dimensional airway volume Ṽₐ(t), taken positive when exhaled, is computed by integration over time of the non-dimensional airway flow rate Q̅ₐ:

$${\tilde{V}}_{a} \left(t\right) = \frac{1}{\left〈T\right〉} {\int }_{0}^{t} {\tilde{Q}}_{a} \left(τ\right) \text{d} τ \text{.} \qquad \text{(13)}$$

### DETERMINATION OF THE BREATHING PATTERN

In step 209 the results of these derivations, for example the pressure signals p_{c}(t) and the Ṽₐ(t)-Q̅ₐ(t) curves may be displayed by the display module 17. An example of a display of the pressure signals p_{c}(t) as a function of time at the start of an NHF therapy is shown in FIG. 5A, while FIG. 5B shows the same relationship at the end of an NHF therapy. An example of a display of the Ṽₐ(t)-Q̅ₐ(t) curves, which bear a strong resemblance to the flow-volume curves that are used in spirometry is shown in FIG. 6.

In this way the method and apparatus of the invention allow a system for NHF therapy to be monitored on the basis of pressure measurements in the air supply to the patient, without any intervention in the patient’s respiratory tract.

Although the invention has been described above by reference to an exemplary embodiment, it will be clear that it is not limited thereto, and may be modified in many ways within the scope of the following claims.

1-29. (canceled)

30. A method for monitoring a system for injecting heated air into a patient, wherein the system includes a source of air, a device for heating the air, a cannula for insertion into nostrils of the patient, a first conduit interconnecting the source and the device, and a second conduit interconnecting the device and the cannula, wherein a pressure in the cannula is derived from a pressure of the air measured at the first location between the source and the cannula.

31. The method of claim 30, wherein a flow rate of the air in the system is determined and wherein the pressure in the cannula is derived from the measured pressure at the first location by compensating for the determined flow rate.

32. The method of claim 31, wherein compensating for the flow rate includes scaling the flow rate by a function of the Reynolds number of the flow of air through the system, and optionally:
wherein the Reynolds number is derived from a pressure differential between the measured pressure at the first location and a measured pressure at a second location upstream of the first location, and wherein a flow restriction is arranged between the first and second locations.

33. The method of claim 30, wherein at least one of:
the derived pressure in the cannula is displayed;
the derived pressure in the cannula is compared to a target pressure to define an error signal and a warning signal is generated when the error signal exceeds a predetermined threshold; and
the derived pressure in the cannula is compared to ambient pressure and a warning signal is generated when the derived pressure in the cannula is below ambient pressure.

34. The method of claim 33, wherein the error signal is used to adjust the flow of air through the system.

35. The method of claim 30, further comprising the steps of:
continuously or periodically measuring a pressure of the air at a first location between the source and the cannula,
determining a flow rate of the air in the system,
deriving a first function representative of a respiratory flow rate of the patient from the measured pressure and the system air flow rate,
deriving a second function representative of a respiratory volume of the patient from the measured pressure and the system air flow rate, and
displaying the first and second functions and/or a variable derived therefrom.

36. The method of claim 35, wherein at least one of:
the pressure is measured during a plurality of respiratory cycles of the patient, and
the second function is derived by integrating the first function.

37. The method of claim 35, wherein the first and second functions are rendered non-dimensional, and optionally:
wherein the first and second functions are rendered non-dimensional by the steps of:
measuring the pressure when no air flows in the system,
measuring the pressure while air is flowing in the system, and
dividing results of the pressure measurements while air is flowing by results of the pressure measurements when no air flows.

38. The method of claim 35, wherein the first function and second function are displayed in a single graph.

39. The method of claim 30, wherein the steps of deriving and displaying the first and second functions and/or deriving, displaying and comparing the pressure in the cannula are performed by a computer.

40. An apparatus for monitoring a system for injecting heated air into a patient, wherein the system includes a source of air, a device for heating the air, cannula for insertion into nostrils of the patient, a first conduit interconnection the source and the device, and a second conduit interconnecting the device and the cannula, wherein a cannula pressure derivation module in communication with a pressure measuring element arranged at a first location between the source and the cannula and configured for deriving a pressure in the cannula from the measured pressure at the first location.

41. The apparatus of claim 40, wherein the cannula pressure derivation module is further in communication with the flow rate determination element and is configured for deriving the pressure in the cannula by compensating the measured pressure at the first location for the flow rate.

42. The apparatus of claim 41, wherein the cannula pressure derivation module is configured for scaling the flow rate by a function of the Reynolds number of the flow of air through the system, and optionally:
wherein the apparatus further comprises a second pressure measuring element arranged at a second location upstream of the first location and a flow restriction arranged between the first and second locations, wherein the cannula pressure derivation module is configured to derive the Reynolds number from a pressure differential between the pressures measured by the first and second pressure measuring elements.

43. The apparatus of claim 40, wherein the display module is in communication with the cannula pressure derivation module and is configured for displaying the derived pressure in the cannula.

44. The apparatus of claim 40, further comprising a comparator in communication with the cannula pressure derivation module and configured for comparing the derived pressure in the cannula to a target pressure to define an error signal and for generating a warning signal when the error signal exceeds a predetermined threshold, and optionally:
wherein the comparator is configured for comparing the derived pressure in the cannula to ambient pressure and for generating a warning signal when the derived pressure in the cannula is below ambient pressure.

45. The apparatus of claim 44, further comprising a flow control member arranged between the source and the cannula and in communication with the comparator to control the flow through the system on the basis of the error signal.

46. The apparatus of claim 40, further comprising:
a pressure measuring element arranged at a first location between the source and the cannula and configured for continuously or periodically measuring a pressure of the air at the first location,
a flow rate determination element for determining a flow rate of the air in the system,
a first derivation module in communication with the pressure measuring element and the flow rate determination element, and configured for deriving a first function representative of a respiratory flow rate of the patient from the measured pressure and the system air flow rate,
a second derivation module in communication with at least one of the pressure measuring element, the flow rate determination element and the first derivation module, and configured for deriving a second function representative of a respiratory volume of the patient from the measured pressure and the system air flow rate, and
a display module in communication with the first and second derivation modules and configured for displaying the first and second functions and/or a variable derived therefrom.

47. The apparatus of claim 46, wherein at least one of:
the pressure measuring element is configured for measuring the pressure during a plurality of respiratory cycles of the patient; and
the second derivation unit is in communication with the first derivation unit and is configured for integrating the first function to derive the second function.

48. The apparatus of claim 46, wherein at least one of the first and second derivation modules is configured to render the first and second functions non-dimensional, and optionally:
wherein the pressure measuring element is configured for measuring the pressure when no air flows in the system and for measuring the pressure while air is flowing in the system, and wherein the first derivation unit is configured for dividing results of the pressure measurements while air is flowing by results of the pressure measurements when no air flows.

49. The apparatus of claim 46, wherein the display module is configured to display the first and second functions in a single graph.
